(19) United States
(12) Patent Application Publication
(10) Pub. No.: US 2008/0300626 A1
(43) Pub. Date: Dec. 4, 2008

(54) ANCHOR SUTURE DEVICE

(76) Inventor: Bert A. Silich, Harrison Township, MI (US)

Correspondence Address: BROOKS KUSHMAN P.C., 1000 TOWN CENTER, TWENTY-SECOND FLOOR, SOUTHFIELD, MI, 48075, US

(21) Appl. No.: 11/756,803

(22) Filed: Jun. 1, 2007

(51) Int. Cl. A61B 17/068

(52) U.S. Cl. 606/219; 606/232

(57) ABSTRACT

An apparatus 10 and method for attaching a medical device 12 to a patient is provided. The apparatus 10 includes an applicator tool 50 containing at least one staple 20. The applicator tool 50 is adapted to insert each staple into the patient. At least some of the staples 20 are adapted to be retained by the patient upon insertion. Most of the staples 20 have at least one attached suture 24. The suture 24 has a first end and a second end. The first end is attached to the staple. The second end in some embodiments has means for stiffening formed thereon.

### BACKGROUND OF THE INVENTION

1. Field of the Invention

The present invention relates to the attachment or anchoring of medical devices, such as catheters and the like, to a patient during medical procedures—particularly in a turbulent or hostile environment—and more particularly to a method and apparatus to quickly and easily attach a medical device to a patient.

2. Background Art

During medical procedures, it often becomes necessary to attach various medical devices to a patient. The types of devices used in the medical field vary with their desired function, size, and the duration over which the device is to be attached to the patient. Frequently during medical procedures and often in emergency medicine, the device must be attached quickly and in a precise location.

One conventional method of attachment is to use a suture attached to a needle. The needle is threaded through the patient and then through an attachment tab on the medical device. The needle must then be removed from the suture and the suture tied off to secure the device to the patient. While this method has been used effectively for quite a long time, there is always the danger of an inadvertent sticking or puncturing someone in the medical team and the risks associated with disposing the used sharp needle. An additional difficulty is that the end of the suture must be long enough to provide the attachment and yet not become encumbering. Frequently, an additional blade must be brought into the area to cut off the excess length of the suture, thereby adding further to the risk of injury.

### SUMMARY OF THE INVENTION

The method disclosed in the present invention involves steps which enable a medical device to be attached to a patient without using a needle in combination with a thread or suture material.

The device described herein is an anchor suture device for anchoring a suture to a patient and providing an attachment point for a medical device. The anchor suture device in one embodiment is a sterile single use applicator tool that contains one or more staples that may be selectively inserted into a patient. Each of the staples has an attached suture for securing the medical device. Preferably, at least some sutures also have a stiffened end to facilitate threading the suture through an attachment on the medical device for anchoring it in relation to the patient.

In one embodiment, the applicator tool may be similar to a standard medical stapler that is modified to accept a small number of staples and a storage magazine to store the staples and sutures. A guarded cutting edge for cutting off any excess suture may also be associated with the applicator tool.

The anchor suture device can be usefully provided in a unitary kit form.

One purpose of the invention is to allow easier, quicker, and safer securing of medical devices, such as central venous catheters, arterial lines, Swan-Ganz catheters, chest tubes, temporary percutaneous gastric (PEG) tubes, percutaneous cystomy catheters, peripheral IV lines, and other like items to a patient without necessitating the use of needles.

Another advantage of the anchor suture device is to reduce the number of unguarded “sharps” at and around a patient in an operating theater or in an environment that may vibrate, be bumpy, or dirty, such as on an aircraft or rotorcraft, or in a combat zone. Thus, the invention is particularly useful in emergency situations or in unsteady, dirty, wet environments such as aircraft, ambulances or in combat zones. This in turn reduces the risk of persons inadvertently puncturing themselves or others with a needle while trying to attach a suture to a patient or cutting themselves or others with a scalpel while cutting off the excess suture material.

Yet another advantage of the anchor suture device to provide a device that is both faster and easier to use when attaching a medical device to a patient.

The disclosed invention also has utility in the veterinary and pediatric medical fields.

### BRIEF DESCRIPTION OF THE DRAWINGS

FIG. 1 is a perspective view showing an anchor suture device attached to a patient for locating and holding a medical device in a fixed location;

FIG. 2 is a perspective view showing a prior art procedure for attaching a medical device to a patient using a needle and a suture or thread material, thereby locating and holding the medical device in a fixed location;

FIG. 3A is a close up perspective view showing a staple of one embodiment of the anchor suture device with an attached suture;

FIG. 3B is a close up exploded perspective view of the embodiment of FIG. 3A showing a staple of the anchor suture device with an attached suture;

FIG. 4 is a perspective view of one embodiment of a suture retainer;

FIG. 5 is a perspective view of another embodiment of a suture retainer;

FIG. 6A depicts an embodiment of a staple used with the anchor suture device;

FIG. 6B depicts the staple of FIG. 6A with the suture retainer removed;

FIG. 6C depicts a staple with inwardly facing ends for use with the anchor suture device showing the suture retainer and the suture;

FIG. 7 is a top view of a staple with an attached suture used in the anchor suture device;

FIG. 8 is an end view of a staple with attached suture used in the anchor suture device;

FIG. 9 is a side view of a staple with attached suture used in the anchor suture device showing the staple in a clinched form;

FIG. 10 is a side elevation view of a prior art needle with attached suture;

FIG. 11 is a side elevation view of an applicator tool, partially cut away to show the storage of the suture;

FIG. 12 is an end view of the applicator tool for the storage of sutures;

FIG. 13 is a top view of the applicator tool showing the storage of sutures and staples;

FIG. 14A is an exploded view of another embodiment of a staple showing the suture retainer separated from the staple;

FIG. 14B is a perspective view showing the suture retainer detached from the staple;

FIG. 15 is a perspective view of an alternate embodiment of a staple-suture retainer combination;

FIG. 16 is a perspective view of another alternate embodiment of a staple; and

FIG. 17 depicts an alternate embodiment of a staple around which a loop of suture is attached.

### DETAILED DESCRIPTION OF THE PREFERRED EMBODIMENT

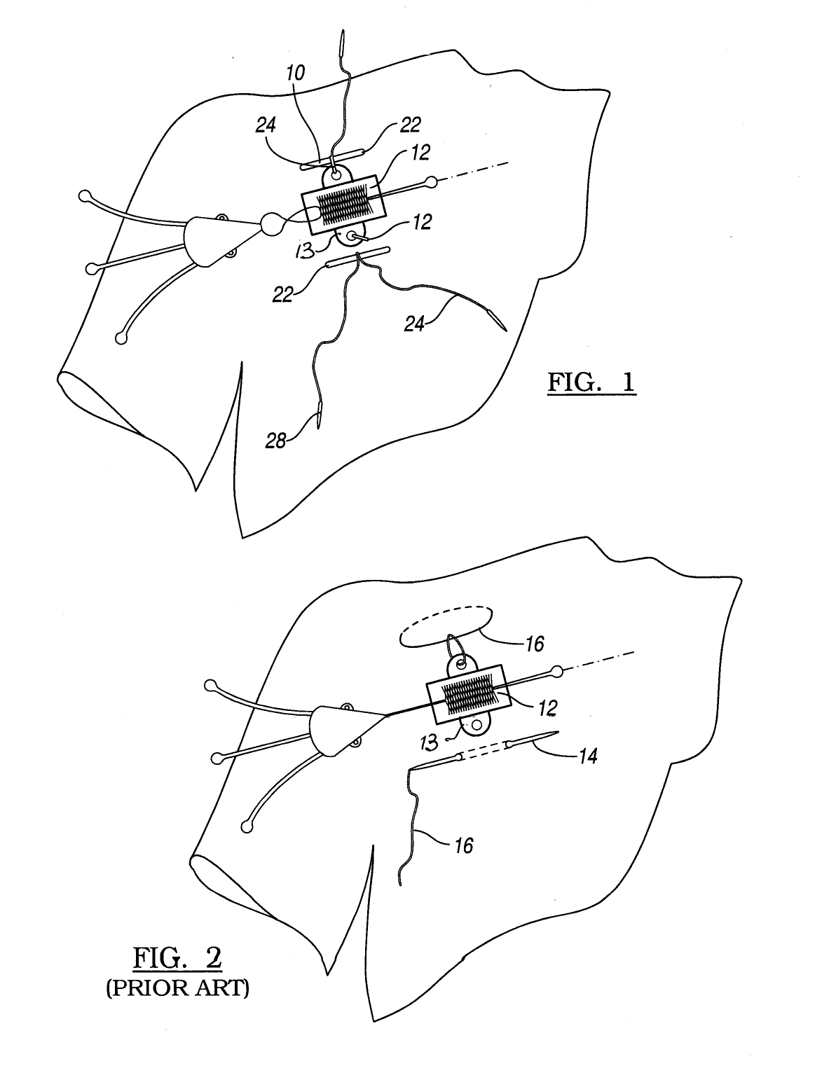

FIG. 1 shows an anchor suture device 10 for anchoring a medical device 12 (such as a catheter assembly) to a patient. A staple 22 is placed into the patient and preferably is clinched to retain the suture 24 in a selected location. The medical device 12 is then attached to the staple 22 by threading the suture through an opening in an attachment tab 13 of the medical device. A suture anchor 10 is shown above the medical device 12 with one or more sutures 24 attached to the device 12. Below the medical device 12, the suture 24 is attached to staple 22, but not yet tethered to the medical device 12.

FIG. 2 is similar to FIG. 1, but shows a conventional attachment technique using a needle 14 and suture 16. The medical device 12 is traditionally attached using a conventional suture 16 that is attached by passing a needle 14 through the skin and looping the suture 16 through the attachment tab 13 of the medical device 12 before tying off the suture 16.

As used herein, the term “staple” connotes a short piece of malleable material that can be bent so as to bind together portions of the skin or underlying tissue or bone or the like by driving the ends therethrough and optionally clinching one or both of the ends therein. If desired, the staple can be shaped similarly to a U-shaped length of wire or surgical metal or plastic with pointed ends for driving into the anatomical surface so that, for example, a suture or suture-like material can be attached. If desired, the staple material can be formed from a thin piece of material in the shape of a square bracket with legs that are driven into an anatomical site and flattened, thereby serving as a fastening.

In FIGS. 1-2, after the suture 24, 16 has been passed through the attachment tab 13, the suture 16, 24 is drawn snug and tied off. The tie-off is typically performed by forming one or more overhand knots in the suture 16, 24. After the suture 16, 24 has been tied off, the excess suture is removed—typically, by cutting off the end of the suture 16, 24 using a scalpel. In light of the approaches that are exemplified by those depicted in FIG. 2, it would be desirable to avoid using a needle and thread to secure the medical device 12 to the patient. This need is particularly felt in hostile environment, whether it be in a combat zone or in a medical evacuation helicopter or other environments that may be adverse.

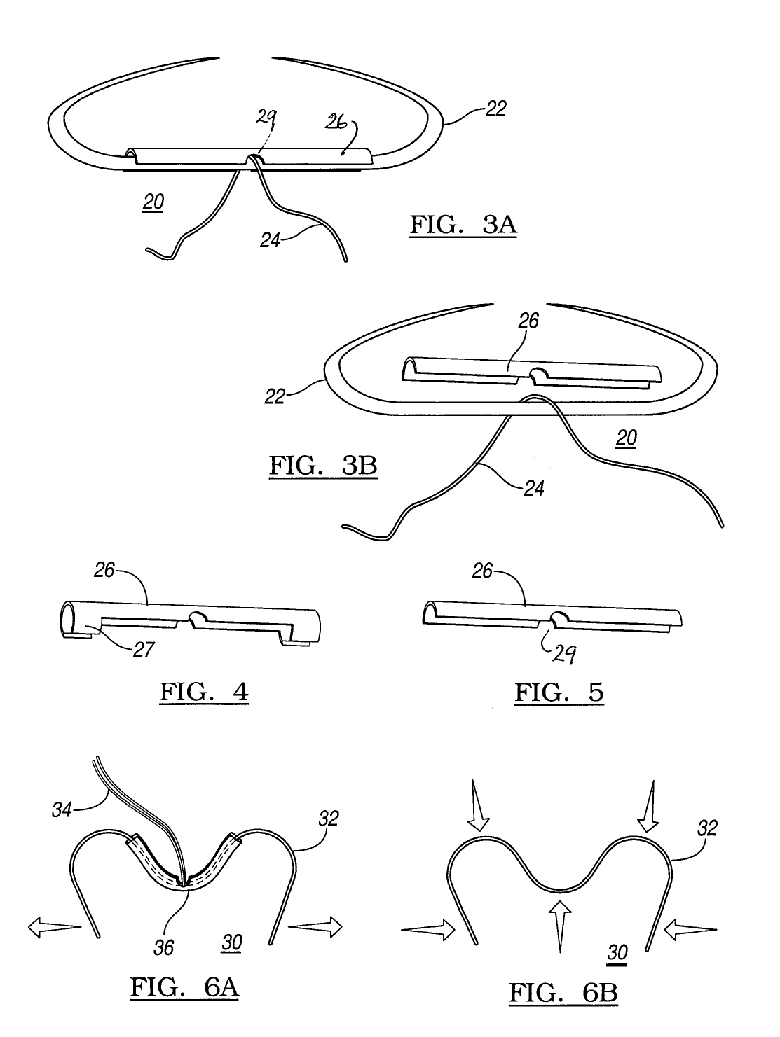

As illustrated in FIGS. 1 and 3-9, the staples 22 used with the anchor suture device 10 may facilitate attachment of a suture 24. For example, FIG. 3A shows a staple 22 where the suture 24 is attached using a suture retainer 26 that can be separated from the staple 22. As shown in FIG. 3B, the suture retainer 26 is a separate piece that can be placed over the shank of the staple 22 to retain the suture 24 in a selected location in relation to the staple 22.

The staples 22 may be fabricated from the same materials as conventional medical staples. They should have the properties of being non-reactive and non-allergenic to tissue and possess sufficient strength to be inserted into a patient and subsequently removed without breaking or leaving metallic or other residue in the patient. Alternatively, depending on the application, the suture may be formed with a thin, malleable wire that may or may not be coated with a sterile substance such as nylon, plastic, or the like. One advantage of using a coated or uncoated thin wire-like material—whether plastic or metallic—is that closure or securement can be obtained merely by twisting and/or knotting the suture material. The suture 24 is formed from any suitable conventional suture material. Suitable materials for sutures include natural fibers such as silk or cotton and manmade fibers including, but not limited to, nylon or polyester.

An alternate form of suture retainer 26 is shown in FIG. 4, in which the suture retainer 26 has tabs 27 formed on each end. The tabs 27 may be crimped around the shank of the staple 22 to affix the staple retainer 26 to the shank of the staple, thereby securing the suture 24 in a selected location on the staple 22 through a detent 29 in the retainer 26.

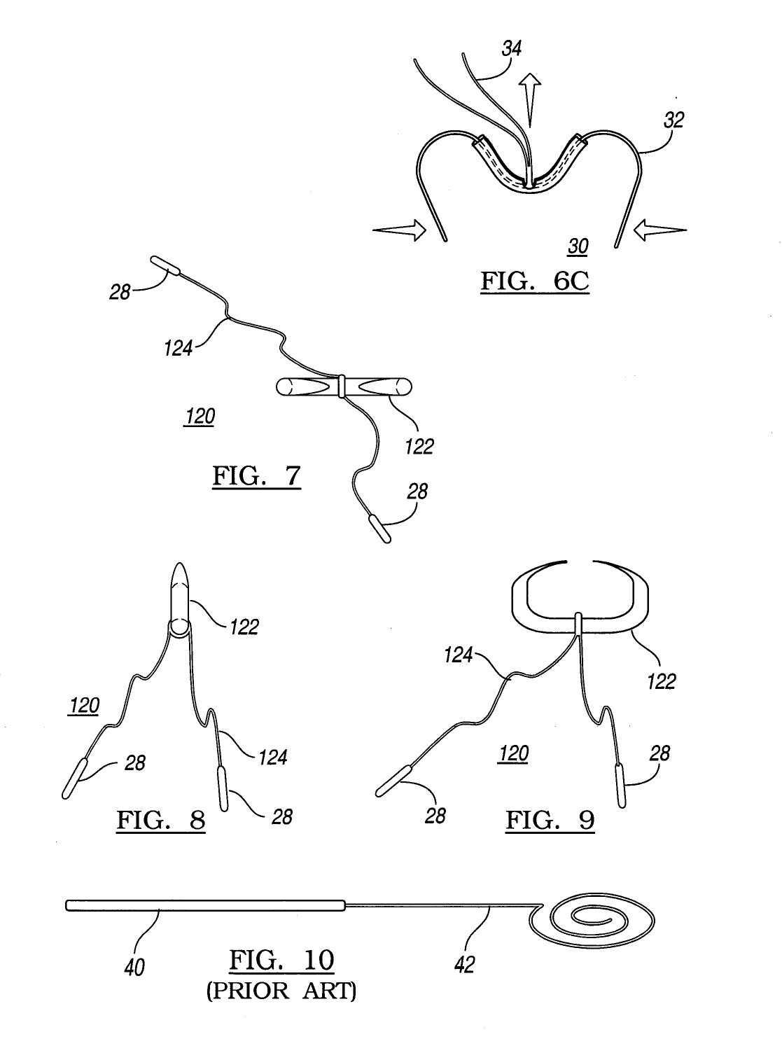

Another embodiment of a staple is shown in FIGS. 6A through 6C. The staple 32 has legs that initially are back-bent (bowlegged). Straightening the shank of the staple 32 causes the legs of the staple to bend inwardly to crimp and better secure the staple 32 to the patient.

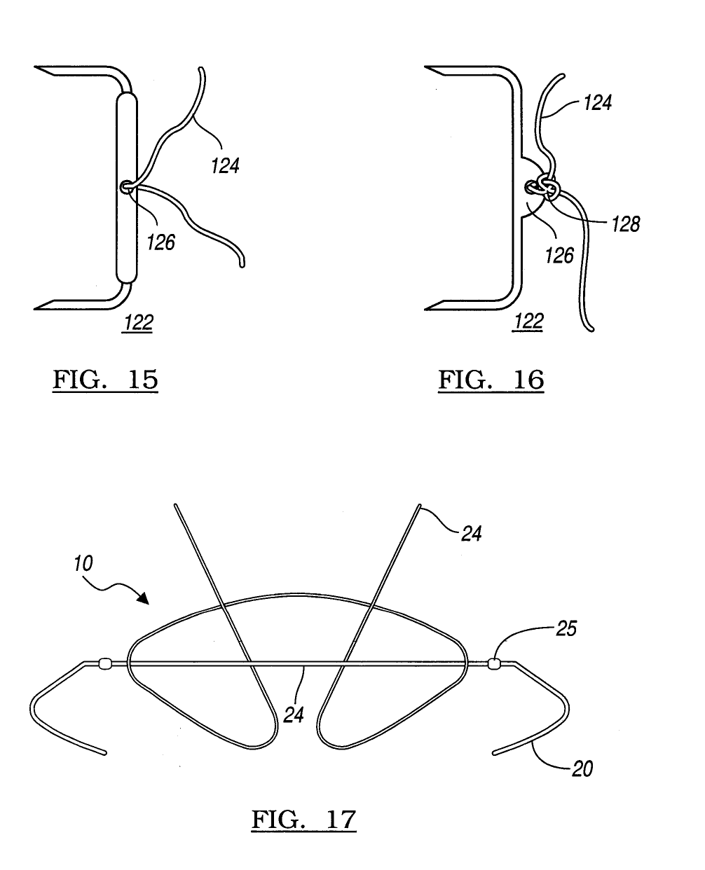

Additional alternate forms of the staples 122 are shown in FIGS. 15 and 16. A separate suture retainer 26 is foregone. In FIG. 16, the suture retainer 126 is incorporated into the staple 122 in the form of an attachment aperture 128.

When a suture 124 is attached to a staple 122 as shown in FIGS. 15 and 16, the suture may be attached at any point along the length of the suture 122 to the suture retainer 128. In this embodiment, attachment is provided by an aperture 128. The suture 124 may be simply passed through the aperture 128. Preferably, the suture is affixed at the aperture by a knot so that the suture 124 cannot be moved through the aperture 128 and is thereby retained for the ease of tying the suture 24 to the medical device.

At least some of the sutures 24 (FIGS. 3A) are attached to or sit astride a shank of a staple 22. At least one end of the suture extends from the detent or attachment point 29. When a suture 24 is attached to a staple 22 such as the staple 22 (FIG. 3A), the suture 24 is attached approximately at its midpoint with the two ends extending therefrom. The suture 22 may be attached by passing the suture 22 around the shank of the staple 22 and retaining the suture 22 in the desired location by placing the suture retainer 26 over the suture 22. Alternately, the suture 22 may be tied or looped around the shank of the staple 22 before the suture retainer 26 is placed on the shank of the staple 22. If the method illustrated in FIG. 17 is practiced, the suture 24 may be passed around the shank of the staple 20 without deploying a suture retainer 26. Optionally, as illustrated in FIG. 17, one or more stops 25 may be provided on the shank of the staple in order to limit travel of the suture 24. In some circumstances, if the suture 24 were allowed to pass beyond a stop 25 toward a side leg of the suture and apply a tensile force thereto, the staple 20 may become unseated. In FIG. 17, there is depicted an alternate embodiment of the anchor suture device 10. It includes a staple 20 that includes beaded portions 25 that constrain movement of the suture 24 along a shank portion 27 of the staple 20. It is appreciated that the term “dimpled portions” (or “means for preventing”) includes any portions that may be raised or in some other fashion serve to prevent the suture 24 loop from sliding away from a central portion or shank 27 of the staple 20.

Regardless of the form of the staple 22, 122 the suture 24, 124 in several embodiments terminates in stiffened ends 28 (FIGS. 7-9). The stiffened ends 28 are provided to assist with threading of the suture 22 through the attachment tabs 13 of the medical device 12. The stiffened ends 28 of the suture 24 may be discrete stiffeners affixed to the respective ends of the suture, or may be a non-toxic stiffening agent impregnating the suture 24, such as wax or a non-toxic polymer. As used herein, the term “wax” means a low-melting organic mixture or compound of high molecular weight that is solid at room temperature. It is generally similar in composition to fats and oils, except that it contains no glycerides. The wax may include hydrocarbons, esters of fatty acids and alcohols. Examples include animal waxes (beeswax), lanolin, shellac wax; vegetable waxes; and mineral waxes. Synthetic waxes include ethylenic polymers and polyol ether-esters, chlorinated napthalenes, and hydrocarbons.

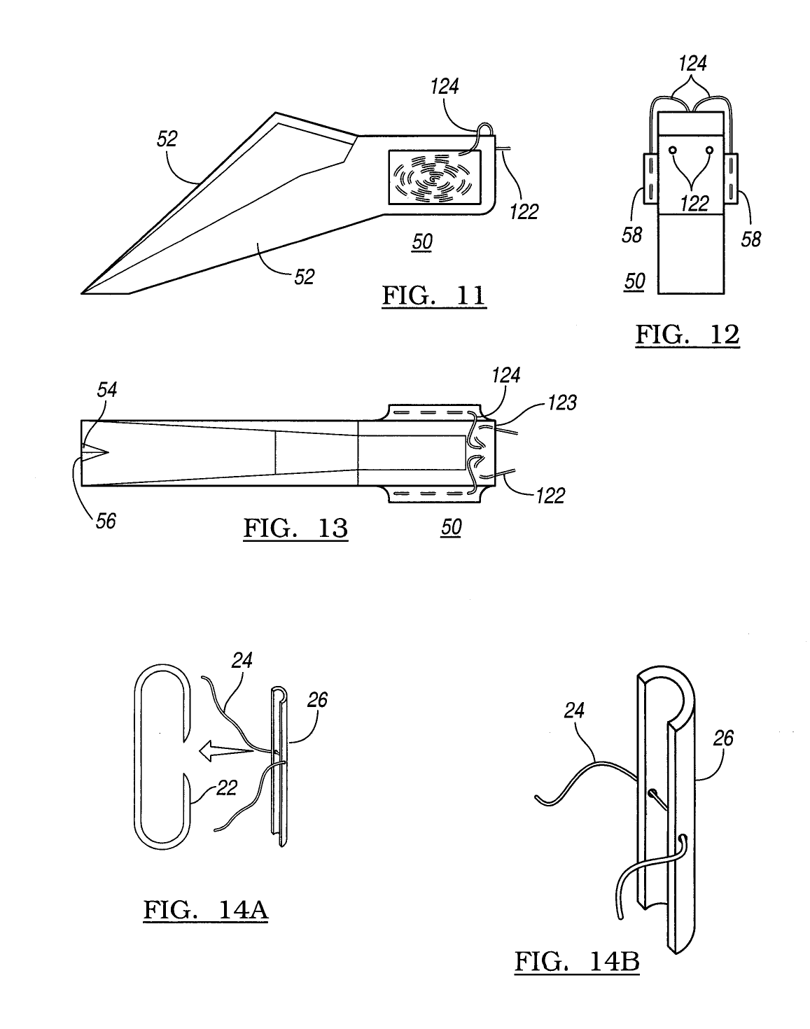

Optionally, an applicator tool 50 (FIGS. 11-13) is provided. In one embodiment, it includes suture-containing magazines 58 along the sides. The magazines 58 contain the sutures 24 and are adapted to allow a suture 24 to be ensnared by each staple 22 as it is expelled from the tool 50. The suture magazines 58 are shown for convenience as being located in one embodiment on each side of the applicator tool 50. It is understood that the suture magazines 58 may be located in any convenient location on the applicator tool 50. Likewise, each of the suture magazines 58 is shown containing one length of suture material 124, attached to one staple 22. It is understood that each suture magazine 58 may be designed to contain multiple sutures 124. In other embodiments, there may optionally be an integrated shielded cutting edge 56 for cutting the suture 16.

In one embodiment (FIG. 13), one magazine may house a spool that accommodates a length of a suture material. A leading end of that material is threaded through a leading or applicator face of the tool 123 before the length is taken up by a spool that is accommodated by the other magazine. In one embodiment, some tension is applied to the suture material by incorporating a spring-loaded feature.

Effectively, before it is ejected, the staple 122 in one embodiment sits astride the length of suture. When the staple is expelled, it draws a length of suture with it as one or both of the spools unwind. In some embodiments, a severing mechanism such as a knife is incorporated in the tool 50 to permit safe and effective cutting of a desired length of suture.

The applicator tool 50 (FIGS. 11-13) has a handle 52 for holding by the operator and actuation. An actuator 53 forms part of the handle 52 so that movement of the actuator 53 expels a staple 122 from the applicator tool 50 and therefore the insertion of the staple 122 into a patient.

The applicator tool 50 may have a guarded cutting edge 56. Also, as shown in FIG. 13, the cutting edge 56 is shown contained in and guarded by a v-shaped groove 54 formed in the handle 52 of the applicator tool 50. While the cutting edge 56 and guard notch 54 are illustrated being on the end of the handle 52 of the applicator tool 50, it is understood that the cutting edge 56 and guard groove 54 may be located at any suitable location on the applicator tool 50. The cutting edge 56 may be formed of any suitable material including various metals on polymers. It is preferred, however, that the cutting edge 56 be formed from sharpened stainless steel owing to the non-corrosive and autoclavable properties of stainless steel.

In practical terms, the doctor, nurse, or paramedic selects a location for the medical device 12 on the patient. The user would then select an applicator tool 50 and locate the applicator tool 50 to insert a staple 22 in a desired location. By actuating the actuator 53 on the handle 52 of the applicator tool 50, a staple 22 is ejected from the applicator tool 50 and injected and secured in the patient. The applicator tool 50 is then removed from the site, withdrawing the suture 14 from the magazine 58 and extending the suture 24 from the staple 22. The stiffened ends 28 of the suture 24 are taken and passed through the attachment tabs 13 of the medical device 12 and secured to secure the medical device 12 in the selected location.

The remaining ends of the suture 22 may then be cut off using the cutting edge 56 on the applicator tool 50. The cut suture 22 ends may then be disposed of in a suitable manner. When multiple staples 22 are required to secure a medical device 12, the procedure is repeated until sufficient staples 22 have been placed to secure the medical device 12. When the medical device 12 is removed, the sutures 22 may be cut and the staples 24 removed using conventional methods.

The previous discussion has assumed that the anchor suture method 10 would use two staples 22 (e.g., FIG. 1) to attach one medical device 12 to a patient and that the materials deployed would be disposed of after use. It is understood that in some applications, that the anchoring method may call for applying only one staple that will suffice to anchor a medical device 12 that requires only one anchoring point.

Alternatively, the anchoring technique may require more than two staples. A multi-staple approach is intended for use in a medical procedure that would require multiple medical devices 12.

Alternative applications of the disclosed invention include use with internal staples and absorbable sutures.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method for attaching a medical device to a patient comprising the steps of:
(a) placing an applicator tool containing at least one staple on a selected site on the patient;
(b) actuating the applicator tool to insert a staple with an attached suture into the selected site;
(c) extending the suture from the staple;
(d) passing an end of the suture through an attachment tab of the medical device, thereby attaching the device to the staple; and
(e) securing the suture with respect to the medical device.

2. The method as described in claim 1 further comprising the step of inserting a plurality of staples into a plurality of selected sites on the patient.

3. The method as described in claim 1 further comprising the step of providing a suture retainer for at least some of the at least one staple for retaining and locating the suture.

4. The method as described in claim 1 further comprising the step of providing a bent-back portion in at least some of the at least one staple, which upon strengthening the staple causes its legs to bend inwardly and crimp, thereby promoting securement of the staple to the patient.

5. An apparatus for attaching a medical device to a patient, the apparatus comprising:
an applicator tool having at least one staple, the applicator tool being adapted to insert a staple into the patient, at least some of the one or more staples being adapted to be retained by the patient upon insertion and having at least one suture attached to the at least one staple.

6. The apparatus as described in claim 5 further comprising a cutting edge for cutting a suture, the cutting edge being provided on the applicator tool.

7. The apparatus as described in claim 6 wherein the cutting edge is inset from a surface of the applicator tool.

8. The apparatus as described in claim 7 wherein the cutting edge is located within a narrow protecting groove defined by the applicator tool.

9. The apparatus as described in claim 5 wherein the applicator tool contains two staples.

10. The apparatus as described in claim 5 wherein the applicator tool includes one or more suture magazines, a handle, and an actuator for expelling the at least one staple.

11. A kit for attaching a medical device to a patient, the kit comprising:
an applicator tool including a suture magazine, a handle, an actuator and a cutting edge;
a length of suture material;
one or more staples; and
a plurality of suture retainers, each retainer being adapted to be retained by a staple.

12. The apparatus as described in claim 11 wherein at least some of the one or more staples are provided with bowed legs.

13. The apparatus as described in claim 11 wherein the suture retainer comprises an elongate semi-tubular retainer that is detachably attachable to a staple, the suture retainer retaining and locating the suture.

14. The apparatus as described in claim 11 wherein the suture retainer has a tab located on at least one end of the suture retainer.

15. The apparatus as described in claim 8 where a tab includes a crimped portion that engages a shank of one of the staples to affix the suture retainer to the shank of the staple.

16. The apparatus as described in claim 5 wherein at least some of the at least one staple initially include a bent-back portion which upon straightening the staple causes its legs to bend inwardly and crimp, thereby promoting securement of the staple to the patient.

17. The apparatus as described in claim 5 further comprising an aperture formed in the at least one staple for receiving a suture therethrough.

18. The apparatus as described in claim 5 further including means for stiffening that comprises a material which impregnates at least part of the suture.

19. The apparatus as described in claim 18 wherein the material includes a wax selected from the group consisting of a natural wax, a synthetic wax and combinations thereof.

20. The apparatus as described in claim 18 wherein the means for stiffening includes a stiffener affixed to a portion proximate an end of the suture.

21. The apparatus as described in claim 5 wherein the one or more staples include one or more internal staples and the at least one attached suture is formed from an absorbable medium.

22. The apparatus as described in claim 5 wherein the one or more staples includes means for preventing the sliding of a suture along the staple.

23. The apparatus as described in claim 11 wherein the plurality of suture retainers includes one or more eyelets through which the suture is threaded.

24. The apparatus as described in claim 11 wherein the plurality of suture retainers defines one or more notches that guide movement of the suture.
